(19) United States
(12) Patent Application Publication
(10) Pub. No.: US 2007/0190159 A1
(43) Pub. Date: Aug. 16, 2007

(54) Polyamino acid-based insulin preparation

(75) Inventors: Kasper Huus Andreasen, Kobenhavn V (DK); Per Balschmidt, Espergaerde (DK); Lone Kimer, Farum (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/786,079

(22) Filed: Apr. 10, 2007

Related U.S. Application Data
(63) Continuation of application No. 11/145,208, filed on Jun. 3, 2005.
Application No. 11/145,208 is a continuation of application No. 10/384,105, filed on Mar. 7, 2003, now abandoned.

(30) Foreign Application Priority Data
Mar. 7, 2002 (DK) PA 2002 00349

(51) Int. Cl. A61K 38/28; A61K 9/14

(52) U.S. Cl. 424/489; 514/3

(57) ABSTRACT

This invention relates to sustained release preparations of insulin comprising poly-amino acid particles, insulin and one or more preservative agents, and a method of preparing such preparations.

### CROSS-REFERENCE TO RELATED APPLICATIONS

This application is a continuation of U.S. application Ser. No. 11/145,208 filed Jun. 3, 2005 (published as US-2005-0233968-A1), which is a continuation of U.S. application Ser. No. 10/384,105 filed Mar. 7, 2003 and claims priority under 35 U.S.C. 119 of Danish Application No. PA 2002 00349 filed Mar. 7, 2002 and U.S. Application No. 60/363,136 filed Mar. 8, 2002, the contents of which are fully incorporated herein by reference.

### FIELD OF THE INVENTION

This invention relates to sustained release preparations of insulin comprising poly-amino acid particles, insulin and one or more preservative agents, and a method of preparing such preparations.

### BACKGROUND OF THE INVENTION

Diabetes is a general term for disorders in man having excessive urine excretion as in diabetes mellitus and diabetes insipidus. Diabetes mellitus is a metabolic disorder in which the ability to utilize glucose is more or less completely lost. About 2% of all people suffer from diabetes.

Since the introduction of insulin in the 1920's, continuous strides have been made to improve the treatment of diabetes mellitus. To help avoid extreme glycemia levels, diabetic patients often practice multiple injection therapy, whereby insulin is administered with each meal.

In the treatment of diabetes mellitus, many varieties of insulin preparations have been suggested and used, such as regular insulin, Semilente® insulin, isophane insulin, insulin zinc suspensions, protamine zinc insulin, and Ultralente® insulin. As diabetic patients are treated with insulin for several decades, there is a major need for safe and life quality improving insulin preparations. Some of the commercial available insulin preparations are characterized by a fast onset of action and other preparations have a relatively slow onset but show a more or less prolonged action. Fast acting insulin preparations are usually solutions of insulin, while retarded acting insulin preparations can be suspensions containing insulin in crystalline and/or amorphous form precipitated by addition of zinc salts alone or by addition of protamine or by a combination of both. In addition, some patients are using preparations having both a fast onset of action and a more prolonged action. Such a preparation may be an insulin solution wherein protamine insulin crystals are suspended. Some patients do themselves prepare the final preparation by mixing an insulin solution with a suspension preparation in the ratio desired by the patient in question.

Protracted insulin compositions are well known in the art. Thus, one main type of protracted insulin compositions comprises injectable aqueous suspensions of insulin crystals or amorphous insulin. In these compositions, the insulin compounds utilised typically are protamine insulin, zinc insulin or protamine zinc insulin.

Certain drawbacks are associated with the use of insulin suspensions. Thus, in order to secure an accurate dosing, the insulin particles must be suspended homogeneously by gentle shaking before a defined volume of the suspension is withdrawn from a vial or expelled from a cartridge. Also, for the storage of insulin suspensions, the temperature must be kept within more narrow limits than for insulin solutions in order to avoid lump formation or coagulation.

Human insulin consists of two polypeptide chains, the so-called A and B chains which contain 21 and 30 amino acids, respectively. The A and B chains are interconnected by two cystine disulphide bridges. Insulin from most other species has a similar construction, but may not contain the same amino acids at the positions corresponding in the chains as in human insulin.

The development of genetic engineering has made it possible easily to prepare a great variety of insulin compounds being analogous to human insulin. In these insulin analogues, one or more of the amino acids have been substituted with other amino acids which can be coded for by the nucleotide sequences. As human insulin, as explained above, contains 51 amino acid residues, it is obvious that a large number of insulin analogues are possible and, in fact, a great variety of analogues with interesting properties have been prepared. In human insulin solutions with a concentration of interest for injection preparations, the insulin molecule is present in associated form as a hexamer (Brange et al. Diabetes Care 13, (1990), 923-954). After subcutaneous injection, it is believed that the rate of absorption by the blood stream is dependent of the size of the molecule, and it has been found that insulin analogues with amino acid substitutions which counteract or inhibit this hexamer formation have an unusual fast onset of action (Brange et al.: Ibid). This is of great therapeutic value for the diabetic patient.

Pharmaceutical preparations which are based on analogues of human insulin have e.g. been presented by Heinemann et al., Lutterman et al. and Wiefels et al. at the “Frontiers in Insulin Pharmacology” International Symposium in Hamburg, 1992.

Furthermore, U.S. Pat. No. 5,474,978 discloses a rapid acting parenteral preparation comprising a human insulin analogue hexamer complex consisting of six monomeric insulin analogues, zinc ions and at least three molecules of a phenolic derivative.

Normally, insulin preparations are administered by subcutaneous injection. What is important for the patient, is the action profile of the insulin preparation which is the action of insulin on the glucose metabolism as a function of the time from the injection. In this profile, inter alia, the time for the onset, the maximum value and the total duration of action are important. A variety of insulin preparations with different action profiles are desired and requested by the patients. One patient may, on the same day, use insulin preparations with very different action profiles. The action profile requested is, for example, depending on the time of the day and the amount and composition of any meal eaten by the patient.

Equally important for the patient is the chemical stability of the insulin preparations, especially due to the abundant use of pen-like injection devices such as devices which contain Penfill® cartridges, in which an insulin preparation is stored until the entire cartridge is empty. This may last for at least 1 to 2 weeks for devices containing 1.5-3.0 ml cartridges. During storage, covalent chemical changes in the insulin structure occur. This may lead to formation of molecules which are less active and potentially immunogenic such as deamidation products and higher molecular weight transformation products (dimers, polymers, etc.). A comprehensive study on the chemical stability of insulin is given in by Jens Brange in “Stability of Insulin”, Kluwer Academic Publishers, 1994.

Another way of attaining an injectable protracted insulin preparation is known from U.S. Pat. No. 5,904,936 to Huille et al. This patent discloses delivery vehicles for active principles comprising nano or micrometer sized particles based on polyamino acids. A method of preparing polyamino acids is disclosed in U.S. Pat. No. 5,780,579. Similar polyamino acid particles are known from WO 00/30618 and WO 01/37809. However, a pharmaceutical preparation containing particles according to one of these documents will be susceptible to microbial degradation. Such a preparation will therefore be unsuited for use in multiple-use applications and where a long shelf-life is required. These disadvantages have now been overcome by the present invention which provides an injectable protracted insulin preparation comprising nano or micrometer sized particles based on polyamino acids, and one or more preservative agents.

### SUMMARY OF THE INVENTION

The present invention provides pharmaceutical preparations comprising (i) particles comprising polyamino acids; (ii) an active ingredient that may be insulin, an insulin derivative, an insulin analogue, or combinations thereof; and (iii) one or more preservative agents. The polyamino acids used in practicing the invention (i) are linear with alpha-peptide linkages; (ii) comprise at least two types of recurring amino acids which are identical or different from one another, which may be hydrophobic neutral amino acids (AAN), including, without limitation, Leu, Ile, Val, Ala, Pro, and Phe, and mixtures thereof, or amino acids having an ionisable side chain (AAI) in which at least portion of the AAI amino acid being in ionised form, including, without limitation, Glu, Asp, and mixtures thereof; and (iii) have a weight average molar mass M_{w} of not less than 4000 D.

The polyamino acids include, without limitation, block polyamino acids, for which the ratio AAN/(AAN+AAI) mole ratio is ≅6% and M_{w}≧5500 D, such as, e.g., block polyamino acids for which the ratio AAN/(AAN+AAI) mole ratio is ≧5% and 6500 D≦M_{w}≦200000 D, and statistical polyamino acids, for which the AAN/(AAN+AAI) mole ratio is ≧20% and M_{w}≧210000 D, including, e.g., statistical polyamino acids. In some embodiments, the polyamino acids comprise a single type of comonomer AAN and a single type of comonomer AAI. Preferably, the weight average molar mass M_{w} of the polyamino acids is not less than 5000 D.

Typically, the particles comprise from 0.01% to 25% dry weight of the preparation, such as, e.g., from 0.05% to 10% dry weight. The average particle size is typically between 0.03 and 0.4 μm. In some embodiments, the particles further comprise at least one aggregating agent. In some embodiments, the particles further comprise a hydrophilic block-copolymer of the polyalkylene-glycol type, such as, e.g., polyethylene glycol. The total concentration of polyamino acids is typically not less than 10⁻²% weight/volume, such as, e.g., between 0.05 and 30% weight/volume or 0.5 and 5% weight/volume.

The preservative agents may be one or more of EDTA, bronopol, benzyl alcohol, benzoic acid, phenylmercuric acetate, thimerosal, glycerol (glycerin), imidurea, chlorohexidine, sodium dehydroacetate, o-cresol, m-cresol, p-cresol, chlorocresol, benzyl alcohol, benzalkonium chloride, cetrimide, benzethonium chloride, methylparaben, ethylparaben, propylparaben, or butylparaben. In one embodiment, the preservative agent is one or more phenolic preservatives, including, without limitation, phenol, m-cresol, or a combination of phenol and cresol. The total concentration of the one or more preservative agents may be, without limitation, 20 to 50 mM, such as, e.g., 32 to 48 mM, 36 to 42 mM, or 38 to 40 mM. In one embodiment, the preparation comprises 16 to 24 mM phenol and 16 to 24 mM m-cresol; in another embodiment, 19 to 21 mM phenol and 19 to 21 mM m-cresol.

In some embodiments, the insulin analogue is an analogue of human insulin, including, without limitation (i) an analogue in which position B28 is Asp, Lys, Leu, Val, or Ala and position B29 is Lys or Pro, such as, e.g., an analogue in which position B28 is Asp or Lys, and position B29 is Lys or Pro; or (ii) des(B28-B30), des(B27) or des(B30) human insulin.

In some embodiments, the insulin derivative is a derivative of human insulin having one or more lipophilic substituents, including, without limitation, B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin, and B29-N^{ε}-(ω-carboxyheptadecanoyl) human insulin.

Typically, the concentration of insulin in the preparations of the invention is from 60 to 3000 nmol/ml, such as, e.g., from 240 to 1200 nmol/ml.

The invention also encompasses methods for preparing a pharmaceutical preparation, which are carried out by
1. Mixing a polyamino acid particle solution with an insulin solution;
2. Adding one or more preservative agents;
3. Incubating the mixture; and
4. optionally adjusting the pH of the mixture.

The preservative agent may be added to the preparation after the insulin and polyamino acid solutions are mixed or after completion of incubation.

In another aspect, the invention encompasses methods for treating diabetes, which are carried out by administering to a patient in need of such treatment an effective amount of a preparation comprising (i) particles comprising polyamino acids; (ii) an active ingredient that may be insulin, an insulin derivative, an insulin analogue, or combinations thereof; and (iii) one or more preservative agents.

### DETAILED DESCRIPTION OF THE INVENTION

### DEFINITIONS

The term insulin as used herein refers to any insulin, including, e.g., human insulin or porcine. Preferably, porcine insulin is highly purified naturally produced porcine insulin.

The term human insulin as used herein refers to naturally produced insulin or recombinantly produced insulin. Recombinant human insulin may be produced in any suitable host cell; for example, the host cells may be bacterial, fungal (including yeast), insect, animal or plant cells.

The term insulin analogue as used herein refers to insulin in which one or more amino acids have been deleted and/or replaced by other amino acids, including non-codeable amino acids, or insulin comprising additional amino acids, i.e. more than 51 amino acids.

The term insulin derivative as used herein refers insulin or an analogue thereof in which at least one organic substituent is bound to one or more of the amino acids.

The term phenolic preservative as used herein refers to a chemical compound in which a hydroxyl group is bound directly to a benzene or substituted benzene ring. Examples of such compounds include, but are not limited to, phenol, o-cresol, m-cresol and p-cresol.

This invention relates to sustained release preparations of insulin comprising polyamino acid particles, insulin and one or more preservative agents, and a method of preparing such preparations.

The polyamino acid particles may be prepared according to the disclosure of U.S. Pat. No. 5,904,936, which is hereby incorporated by reference. In one embodiment of the invention, the particles are prepared according to the method described in example 1 of U.S. Pat. No. 5,904,936. In other embodiments of the invention, the particles are prepared according to the methods described in examples 2, 3, 4, 5, 8, 16, 17, or 18 of U.S. Pat. No. 5,904,936. Particles further comprising polyalkylene-glycol are disclosed in WO 02/78677, and may be prepared by a method described e.g. in example 1 or 2 of said reference.

In another embodiment of the invention, the particles are prepared according to the disclosure of WO 00/30618 which is hereby incorporated by reference, e.g. page 9, line 22 to page 10, line 9. In one embodiment of the invention, the particles are prepared according the method described in example 1 of WO 00/30618. In other embodiments of the invention, the particles are prepared according to the methods described in examples 2, 3, or 4 of WO 00/30618.

In another embodiment of the invention, the particles are prepared according to the disclosure of WO 01/37809 which is hereby incorporated by reference, e.g. page 14, line 1 to page 17, line 10. In one embodiment of the invention, the particles are prepared according the method described in example 1 of WO 01/37809. In other embodiments of the invention, the particles are prepared according to the methods described in examples 3, 4, 5, or 6 of WO 01/37809. The particles are based on amphiphilic, linear polyamino acids with alpha-peptide linkages, and comprise at least two different types of recurrent amino acids, i.e. hydrophilic and neutral hydrophobic amino acids, the amino acids within each group being either identical or different. The hydrophilic amino acids are chosen among those with ionisable side chains, with amino acids Glu and Asp in carboxylic or salt form being particularly preferred. The neutral hydrophobic amino acids are chosen from natural neutral amino acids, preferably those belonging to the sub-group comprising Leu, Ile, Val, Ala, Pro, and Phe. The particles are stable at a pH between 4 and 13 in the absence of a surfactant. They have an insulin load factor ranging between 5 and 25% of associated insulin volume relative to the polyamino acid volume. The particles have a mean hydrodynamic diameter between 10 and 150 nm, preferably between 20 and 100 nm. The mean hydrodynamic diameter is measured as described in WO 01/37809 on page 9, line 11 to line 21. The insulin load factor is measured by the method described in WO 01/37809 on page 9, line 23 to page 10, line 7.

The preparation moreover comprises insulin. In one embodiment the insulin is selected from the group consisting of human insulin, an analogue thereof, a derivative thereof, and combinations of any of these.

In another embodiment the insulin is an analogue of human insulin selected from the group consisting of
i. An analogue wherein position B28 is Asp, Lys, Leu, Val, or Ala and position B29 is Lys or Pro; and
ii. des(B28-B30), des(B27) or des(B30) human insulin.

In another embodiment the insulin is an analogue of human insulin wherein position B28 is Asp or Lys, and position B29 is Lys or Pro.

In another embodiment the insulin is des(B30) human insulin.

In another embodiment the insulin is LysB3 GluB29.

In another embodiment the insulin is a derivative of human insulin having one or more lipophilic substituents.

In another embodiment the insulin derivative is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-ThrB29LysB30 human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin. In another embodiment the insulin derivative is B29-N^{ε}-myristoyl-des(B30) human insulin.

The preparation according to the present invention furthermore comprises a preservative agent.

In one embodiment of the invention the preservative agent is selected from the group consisting of EDTA, bronopol (e.g. in a concentration of 0.5 to 5 mM), benzyl alcohol (e.g. in a concentration of 80 to 300 mM), benzoic acid (e.g. in a concentration of 10 to 100 mM), phenylmercuric acetate, thimerosal, glycerol (glycerin), imidurea, chlorohexidine, sodium dehydroacetate, phenolic preservatives such as phenol, o-cresol, m-cresol, p-cresol, or chlorocresol (the latter e.g. in a concentration of 1 to 20 mM), benzalkonium chloride, cetrimide, benzethonium chloride, methylparaben, ethylparaben, propylparaben, or butylparaben (the total concentration of parabenes being e.g. in the range from 0.02 to 0.3% (W/V)), or a combination of one or more of these.

In another embodiment the preservative agent is one or more phenolic preservatives.

In another embodiment the preservative is phenol or m-cresol, or a combination of both.

In another embodiment the total concentration of the one or more phenolic preservatives is 20 to 50 mM.

In another embodiment the total concentration of the one or more phenolic preservatives is 32 to 48 mM.

In another embodiment the total concentration of the one or more phenolic preservatives is 36 to 42 mM.

In another embodiment the total concentration of the one or more phenolic preservatives is 38 to 40 mM.

In another embodiment the concentration of the phenolic preservatives is 16 to 24 mM of phenol and 16 to 24 mM of m-cresol.

In another embodiment the concentration of the phenolic preservatives is 19 to 21 mM of phenol and 19 to 21 mM of m-cresol.

In another embodiment the preparation comprises 30 to 48 mM of m-cresol. In another embodiment the preparation comprises 36 to 42 mM of m-cresol.

In one aspect of the invention the present pharmaceutical preparation is made available to the patient or medical personnel in the form of vials or other medicament containers containing the preparation. In another aspect, the preparation is made available in the form of a cartridge for use in pen injector devices. Such devices may be either disposable or durable. The preparation may also be used in an insulin pump system.

The preparations of this invention may contain further constituents such as a zinc salt, for example zinc chloride, an isotonic agent, for example sodium chloride or glycerol, and a buffer, for example disodium monohydrogen phosphate, in an aqueous medium. The pH of the preparation may furthermore be adjusted, e.g. to a pH value of from about 4 to about 8. In one embodiment of the invention, the pH is adjusted to about pH 7.3.

The amount of polyamino acid particles added to the pharmaceutical preparation of the invention can be determined in two ways. One is described in U.S. Pat. No. 5 904 936, see e.g. example 14. The amount of insulin adsorbed to the polyamino acid particles is calculated as the difference between added insulin and insulin present in dissolved form after filtration of the preparation. In this way, a person skilled in the art will be able to determine by routine experimentation the amount of polyamino acid particles necessary to adsorb a given amount of insulin. Another method is to carry out experiments in an animal model with preparations containing insulin adsorbed to varying amounts of polyamino acid particles. One model is measurement of the blood glucose lowering effect of the product when administered subcutaneously to pigs. The onset and duration of the blood glucose lowering effect is dependent on the amount of poly amino acid particles relative to the amount of insulin; this ratio determines the amount of free insulin, which provides a fast onset, and the amount of insulin adsorbed to the poly amino acid particles, which provides a prolonged effect. Thus, if a fast onset is observed, a surplus of insulin is known to have been present in the preparation.

The invention also relates to a method of treating diabetes in a patient comprising administering to said patient a pharmaceutical preparation comprising polyamino acid particles, insulin and one or more preservative agents, in an amount effective to treat the diabetes.

The pharmaceutical preparation according to the present invention is made in a method which comprises the following steps:
1. Mixing a polyamino acid particle solution obtained as described above with an insulin solution
2. adding one or more preservative agents.
3. Incubating until sufficient insulin is adsorbed to the polyamino acid particles
4. Optionally adjusting pH In one embodiment of the invention, the concentration of the insulin in the final preparation is from 60 to 3000 nmol/ml. In another embodiment, the concentration of the insulin in the final preparation is from 240 to 1200 nmol/ml. In another embodiment, the incubation is carried out at room temperature. In another embodiment, the incubation has a duration of from 1 to 24 hours, preferably 6 to 12 hours. In another embodiment, the preservative agent is added after the mixing of polyamino acids and insulin solution. In another embodiment, the preservative agent is added after the incubation step.

### EXAMPLES

The following examples are intended as non-limiting illustrations of the present invention.

Preservative Efficacy Screening

Six experiments were carried out with preparations containing 50 mg/ml polyamino acid particles and 600 nmol/ml human insulin. Preservative agents were added as shown in table 1.

TABLE 1
Concentration of preservatives in example 1-6

| | Example 1 | Example 2 | Example 3 | Example 4 | Example 5 | Example 6 |
|---|---|---|---|---|---|---|
| m-cresol (mM) | 16 | 19 | 24 | 32 | 32 | 40 |
| Phenol (mM) | 16 | 19 | 24 | 0 | 8 | 0 |

Preservative efficacy was measured as described in Ph. Eur. 4^{th} Edition, 5.1.3: “Efficacy of antimicrobial preservation”. Only one test micro-organism (Staphylococcus Aureus) was used in this case. The result is shown in table 2.

TABLE 2
Concentration (cfu/mL) of Staphylococcus Aureus after incubation
with the preparations and the calculated log reductions

| | Example 1 | Example 2 | Example 3 | Example 4 | Example 5 | Example 6 |
|---|---|---|---|---|---|---|
| Concentration (cfu/ml) at | 2.2 × 10⁵ | 2.2 × 10⁵ | 2.2 × 10⁵ | 2.3 × 10⁵ | 2.3 × 10⁵ | 2.1 × 10⁵ |
| time = 0 h | | | | | | |
| Concentration (cfu/ml) at | 2.1 × 10⁵ | 2.3 × 10⁵ | 5.1 × 10⁴ | 1.9 × 10⁵ | 5.9 × 10⁴ | 1.5 × 10⁴ |
| time = 6 h | | | | | | |
| Concentration (cfu/ml) at | 1.4 × 10⁵ | 6.0 × 10³ | 1.0 × 10¹ | 2.1 × 10² | 1.0 × 10¹ | 1.0 × 10¹ |
| time = 24 h | | | | | | |
| Concentration (cfu/ml) at | 1.2 × 10⁴ | 1.0 × 10¹ | 1.5 × 10¹ | 1.0 × 10¹ | 1.0 × 10¹ | 1.0 × 10¹ |
| time = 48 h | | | | | | |
| Log reduction 24 h | 0.2 | 1.5 | 4.3 | 3.0 | 4.3 | 4.3 |
| Log reduction 48 h | 1.2 | 4.3 | 4.1 | 4.3 | 4.3 | 4.3 |

All patents, patent applications, and literature references referred to herein are hereby incorporated by reference in their entirety.

Many variations of the present invention will suggest themselves to those skilled in the art in light of the above detailed description. Such obvious variations are within the full intended scope of the appended claims

1. A pharmaceutical preparation comprising
i. Particles comprising polyamino acids, wherein said polyamino acids
a. are linear with alpha-peptide linkages,
b. comprise at least two types of recurring amino acids which are identical or different from one another, selected from the group consisting of a hydrophobic neutral amino acid (AAN), and an amino acid having an ionisable side chain (AAI), at least a portion of the AAI amino acid being in ionised form, and
c. Have a weight average molar mass M_{w} of not less than 4000 D;
ii. An active ingredient selected from the group consisting of insulin; an insulin derivative; an insulin analogue; and combinations of any of the foregoing; and
iii. One or more preservative agents.

2. A pharmaceutical preparation according to claim 1, wherein the particles comprise polyamino acids selected from the group consisting of block and statistical polyamino acids, wherein for the block polyamino acids, the ratio AAN/(AAN+AAI) mole ratio is ≧6% and M_{w}≧5500 D, and for the statistical polyamino acids the AAN/(AAN+AAI) mole ratio is ≧20% and M_{w}≧10000 D.

3. A pharmaceutical preparation according to claim 1, wherein the hydrophobic neutral amino acid is selected from the group consisting of Leu, Ile, Val, Ala, Pro, and Phe, and mixtures thereof, and the amino acid having an ionisable side chain is selected from the group consisting of Glu and Asp, and mixtures thereof.

4. A pharmaceutical preparation according to claim 1, wherein the average particle size is between 0.03 and 0.4 μm.

5. A pharmaceutical preparation according to claim 1, wherein the weight average molar mass M_{w} of the polyamino acids is not less than 5000 D.

6. A pharmaceutical preparation according to claim 1, wherein the particles comprise polyamino acids selected from the group consisting of block and statistical polyamino acids and wherein for the block polyamino acids, the ratio AAN/(AAN+AAI) mole ratio is ≧5% and 6500 D≦M_{w}≦200000 D, and for the statistical polyamino acids the AAN/(AAN+AAI) mole ratio is ≧25% and 20000 D≦M_{w}≦500000 D.

7. A pharmaceutical preparation according to claim 6, wherein for the block polyamino acids, 8000 D≦M_{w}≦200000 D, and for the statistical polyamino acids 20000 D≦M_{w}≦150000 D.

8. A pharmaceutical preparation according to claim 1, wherein the particles further comprise at least one aggregating agent.

9. A pharmaceutical preparation according to claim 1, wherein the particles based on polyamino acids further comprise a hydrophilic block-copolymer of the polyalkylene-glycol type.

10. A pharmaceutical preparation according to claim 1, wherein the hydrophilic block-copolymer of the polyalkylene-glycol type is polyethylene glycol.

11. A pharmaceutical preparation according to claim 1, wherein the polyamino acids comprise a single type of comonomer AAN and a single type of comonomer AAI.

12. A pharmaceutical preparation according to claim 1, wherein the concentration of the polyamino acids is not less than 10⁻²% weight/volume.

13. A pharmaceutical preparation according to claim 12, wherein the concentration of the polyamino acids is between 0.05 and 30% weight/volume.

14. A pharmaceutical preparation according to claim 12, wherein the concentration of the polyamino acids is between 0.5 and 5% weight/volume.

15. A pharmaceutical preparation according to claim 1, wherein said one or more preservative agents is selected from the group consisting of EDTA, bronopol, benzyl alcohol, benzoic acid, phenylmercuric acetate, thimerosal, glycerol (glycerin), imidurea, chlorohexidine, sodium dehydroacetate, o-cresol, m-cresol, p-cresol, chlorocresol, benzalkonium chloride, cetrimide, benzethonium chloride, methylparaben, ethylparaben, propylparaben, butylparaben, and combinations of any of the foregoing.

16. A pharmaceutical preparation according to claim 15, wherein the preservative agent is one or more phenolic preservatives.

17. A pharmaceutical preparation according to claim 1, wherein the total concentration of the one or more preservative agents is 20 to 50 mM.

18. A pharmaceutical preparation according to claim 17, wherein the total concentration of the one or more preservative agents is 32 to 48 mM.

19. A pharmaceutical preparation according to claim 18, wherein the total concentration of the one or more preservative agents is 36 to 42 mM.

20. A pharmaceutical preparation according to claim 19, wherein the total concentration of the one or more preservative agents is 38 to 40 mM.

21. A pharmaceutical preparation according to claim 1, wherein the preservative is phenol, m-cresol, or a combination of phenol and cresol.

22. A pharmaceutical preparation according to claim 21, comprising 16 to 24 mM phenol and 16 to 24 mM m-cresol.

23. A pharmaceutical preparation according to claim 22, comprising 19 to 21 mM phenol and 19 to 21 mM m-cresol.

24. A pharmaceutical preparation according to claim 1, comprising 30 to 48 mM m-cresol.

25. A pharmaceutical preparation according to claim 24, comprising 36 to 42 mM m-cresol.

26. A pharmaceutical preparation according to claim 1, wherein the insulin analogue is an analogue of human insulin selected from the group consisting of
iii. An analogue wherein position B28 is Asp, Lys, Leu, Val, or Ala and position B29 is Lys or Pro; and
iv. des(B28-B30), des(B27) or des(B30) human insulin.

27. A pharmaceutical preparation according to claim 26, wherein the insulin analogue is an analogue of human insulin wherein position B28 is Asp or Lys, and position B29 is Lys or Pro.

28. A pharmaceutical preparation according to claim 26, wherein the insulin analogue is des(B30) human insulin.

29. A pharmaceutical preparation according to claim 1, wherein the insulin derivative is a derivative of human insulin having one or more lipophilic substituents.

30. A pharmaceutical preparation according to claim 29, wherein the insulin derivative is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin, and B29-N^{ε}-(ω-carboxyheptadecanoyl) human insulin.

31. A pharmaceutical preparation according to claim 30, wherein the insulin derivative is B29-N^{ε}-myristoyl-des(B30) human insulin.

32. A pharmaceutical preparation according to claim 1, wherein the concentration of insulin is from 60 to 3000 nmol/ml.

33. A pharmaceutical preparation according to claim 32, wherein the concentration of insulin is from 240 to 1200 nmol/ml.

34. A method of preparing a pharmaceutical preparation, said method comprising the steps of
1. Mixing a polyamino acid particle solution with an insulin solution to form a mixture;
2. Adding one or more preservative agents;
3. Incubating the mixture; and
4. Optionally adjusting the pH of the mixture.

35. A method according to claim 34, wherein the preservative agent is added to the preparation after the insulin and polyamino acid particle solutions are mixed.

36. A method according to claim 35, wherein the preservative is added to the preparation after completion of incubation.

37. A method of treating diabetes, said method comprising administering to a patient in need of such treatment an effective amount of a preparation according to claim 1.
